(19) United States
(12) Patent Application Publication
(10) Pub. No.: US 2005/0208652 A1
(43) Pub. Date: Sep. 22, 2005

(54) Isogenic beta-catenin cell lines, and methods of making and using same

(76) Inventor: Todd Waldman, Washington, DC (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 11/104,063

(22) Filed: Apr. 11, 2005

Related U.S. Application Data
(63) Continuation of application No. 10/093,684, filed on Mar. 7, 2002.
(60) Provisional application No. 60/274,393, filed on Mar. 9, 2001.

(51) Int. Cl. C12N 5/08

(52) U.S. Cl. 435/366

(57) ABSTRACT

A set of isogenic cell lines, which includes a first population of cells that express only a wild type β-catenin polypeptide and at least a second population of cells that express only an activated β-catenin polypeptide, is provided. A set of isogenic cells, including a first population of cells that are null for β-catenin expression, and at least a second population of cells that express a wild type β-catenin polypeptide that functions as an activated β-catenin polypeptide in the cells, also is provided. In addition, a recombinant nucleic acid molecule, which includes at least a first linear polynucleotide that is flanked at each end by nucleotide sequences of a β-catenin gene is provided, as is a method of using the recombinant nucleic acid molecule to produce a set of isogenic cell lines as defined above. Also provide is a method of using the set of isogenic cell lines to identify a therapeutic agent that allows selective killing of cells expressing an activated β-catenin polypeptide, but not cells expressing a wild type β-catenin polypeptide is provided.

This application claims the benefit under 35 U.S.C. 119(e) of U.S. Serial NO. 60/274,393 filed Mar. 9, 2001, the entire contents of which is incorporated herein by reference.

This invention was made in part with government support under Grant No. KO 01 CA87828-01 awarded by the National Institutes of Health. The United States government has certain rights in this invention.

### BACKGROUND OF THE INVENTION

1 Field of the Invention

The invention relates generally to cell lines useful for identifying therapeutic agents and, more specifically, to isogenic cell lines, including isogenic cell lines that differ only in containing either a normal β-catenin gene or an activated β-catenin gene, and isogenic cell lines that differ only in the expression or lack of expression of an activated β-catenin polypeptide; to methods and compositions for making such isogenic cell lines; and to methods of using the isogenic cell lines to identify therapeutic agents that allow for selectively killing cells expressing an activated β-catenin polypeptide, but not cells expressing a normal β-catenin polypeptide.

2 Background Information

Despite numerous advances in the diagnosis and treatment of cancer, it remains a major cause of suffering and death in the United States and the world. In the past few years, genetic tests have started to become available, providing a means to detect persons at risk of developing a cancer or to detect cancer in very early stages. Identification of mutations in the BRCA 1 gene, which is associated with breast cancer in women, is an example of such a genetic test. The deciphering of the human genome promises to reveal a great number of additional genes that similarly will be useful for diagnostic procedures.

Methods of treating cancer also have advanced over those available only a generation ago. However, the advances in cancer treatment have been modest, and generally are based on minor advances in established fields. For example, surgery remains a primary treatment for many cancer patients. However, while surgical techniques have advanced due to the availability better instrumentation, including computer assisted instruments, and more precise and accurate methods for examining the region surrounding the excised tumor, the basic approach of debulking the mass of a tumor remains essentially unchanged.

Similarly, chemotherapy remains the treatment of choice for patients suffering from certain types of cancer, including more disseminated forms of cancer. While efforts have been made to identify chemotherapeutic agents that selectively kill cancer cells, but leave normal cells undamaged, most chemotherapeutic agents act by killing cells based on their rate of cell division or based on the ability of normal cells to repair damage to a greater extent than cancer cells. As such, cancer cells, which often divide more rapidly or have less capacity for repairing damage than their normal cell counterparts, are more preferentially killed than their corresponding normal cells. However, many cell types in the body, including red and white blood cell precursors, intestinal epithelial cells, and the like, also divide relatively rapidly and, therefore, are killed by the chemotherapeutic agent, resulting in the nausea, susceptibility to infection, and hair loss that are common and well known side effects of chemotherapy.

Although efforts have been made to identify therapeutic agents that can selectively kill cancer cells, while sparing normal cells, these efforts generally have been based on empirical studies, wherein large numbers of compounds are screened in hopes of identifying a particularly selective agent. The advent of methods for high throughput screening of large numbers of compounds that may be useful for therapeutic purposes has helped in the search for useful therapeutic agents. However, the basic “hit or miss” aspect of such studies essentially are the same as those used since chemotherapeutic agents were first identified. Thus, a need exists for efficient and effective methods to identify a therapeutic agent that selectively kill a diseased cell, while sparing its normal cell counterpart. The present invention satisfies this need and provides additional advantages.

### SUMMARY OF THE INVENTION

The present invention relates to a set of isogenic cell lines, which includes a first population of cells that express only a wild type β-catenin polypeptide, and at least a second population of cells that express only an activated β-catenin polypeptide, wherein at least one of the populations of cells contains a disrupted β-catenin gene, and wherein, except for a nucleotide sequence of a β-catenin gene, the first population of cells and the at least second population of cells are substantially genetically identical. The cells in a set of isogenic cell lines of the invention can be near diploid or diploid. The first population of cells in the set can be hemizygous for a wild type β-catenin gene, which encodes the wild type β-catenin, or the second population of cells in the set can be hemizygous for a mutant β-catenin gene, which encodes the activated β-catenin polypeptide. For example, the cells of the first population of cells can contain a wild type β-catenin gene and a disrupted mutant β-catenin gene, or the cells of the second population of cells can contain a mutant β-catenin gene, which encodes the activated β-catenin polypeptide, and a disrupted wild type β-catenin gene.

A set of isogenic cell lines of the invention can further include at least a third population of cells, which, for example, express a wild type β-catenin polypeptide and an activated β-catenin polypeptide. Such cells can be heterozygous for a wild type β-catenin gene and a mutant β-catenin gene, or can be, for example, hemizygous for a wild type β-catenin gene and genetically modified to contain a polynucleotide encoding an activated β-catenin polypeptide. The cells of the third population of cells also can be null for β-catenin expression, wherein one or both endogenous β-catenin genes is disrupted. The cells comprising a set of isogenic cell lines of the invention can be any cells, including, for example, mammalian cells. In one embodiment, the cells are human cells. In another embodiment, the cells are human cancer cells, for example, HCT 116 human colon adenocarcinoma cell lines.

A set of isogenic cell lines of the invention can include a first population of cells that express a wild type β-catenin polypeptide, wherein the cells of the first population are hemizygous for a wild type β-catenin gene, which encodes the wild type β-catenin polypeptide; a second population of cells that express an activated β-catenin polypeptide, wherein the second population of cells is hemizygous for a mutant β-catenin gene, which encodes the activated β-catenin polypeptide; and a third population of cells that is null for

β-catenin expression. In addition, the set can further include at least a fourth population of cells, wherein the cells express a wild type β-catenin polypeptide and are homozygous for a wild type β-catenin gene; or wherein the cells express an activated β-catenin polypeptide and are homozygous for a mutant β-catenin gene, which encodes the activated β-catenin polypeptide. Alternatively, the set can include a fourth population of cells that express a wild type β-catenin polypeptide and are homozygous for a wild type β-catenin gene; and at least a fifth population of cells that express an activated β-catenin polypeptide and are homozygous for a mutant β-catenin gene, which encodes the activated β-catenin polypeptide.

The present invention also relates to a recombinant nucleic acid molecule, which includes at least a first linear polynucleotide that is flanked at each end by nucleotide sequences of a β-catenin gene. Such a recombinant nucleic acid molecule is designed such that it can be used for insertion, for example, by homologous recombination, into a β-catenin gene in a cell, including into a wild type β-catenin gene, into a mutant β-catenin gene that encodes an activated β-catenin polypeptide, or into both. As such, the recombinant nucleic acid molecule is useful for knocking out ability of the gene to encode a wild type or an activated β-catenin polypeptide or both. The polynucleotide portion of the recombinant nucleic acid molecule can encode a polypeptide such as a selectable marker or reporter molecule, which can facilitate identification and isolation of a cell having the recombinant nucleic acid molecule integrated into its genome.

In one embodiment, a recombinant nucleic acid molecule of the invention lacks a transcriptional promoter element, the polynucleotide encodes a selectable marker, and the flanking β-catenin gene nucleotide sequences are selected such that the sequence encoding the initiator methionine of the selectable marker precisely replaces the sequence encoding the initiator methionine of the target β-catenin gene. In another embodiment, the polynucleotide lacks an initiator methionine residue of an encoded polypeptide, and the nucleotide sequences of the β-catenin gene are selected such that the polynucleotide inserts immediately upstream and adjacent to the initiator methionine codon of the target β-catenin gene. In still another embodiment, the recombinant nucleic acid molecule is contained in a vector. Accordingly, the present invention also provides a targeting vector, which can be used to introduce the recombinant nucleic acid molecule into a target cell.

The present invention further relates to a method of producing a set of isogenic cell lines, which includes a first population of cells that express a wild type β-catenin polypeptide and at least a second population of cells that express an activated β-catenin polypeptide. Such a method can be performed, for example, by introducing a recombinant nucleic molecule of the invention into cells that are heterozygous for a mutant β-catenin gene, which encodes an activated β-catenin polypeptide, and a wild type β-catenin gene, which encodes a wild type β-catenin polypeptide; and selecting a first population of cells, which are derived from a cell containing the recombinant nucleic acid molecule integrated into only the mutant β-catenin gene and express only the wild type β-catenin polypeptide; and at least a second population of cells, which are derived from a cell containing the recombinant nucleic acid molecule integrated into only the wild type β-catenin gene and express only the activated β-catenin polypeptide. A method of the invention also can include selecting at least a third population of cells, which are derived, for example, from a cell containing the recombinant nucleic acid molecule integrated into both the mutant β-catenin gene and the wild type β-catenin gene, wherein said cells are null for β-catenin polypeptide expression.

In one embodiment, the cells that are heterozygous for the mutant and wild type β-catenin genes are mammalian cells, particularly human cells. In another embodiment, the cells human cells are human cancer cells, for example, HCT 116 human colon adenocarcinoma cell lines. The recombinant nucleic acid molecule can be integrated into the cell genome by homologous recombination, and the polynucleotide in the recombinant nucleic acid molecule can encode a polypeptide, which, for example, can confer a detectable phenotype on the cell. The detectable phenotype can be any phenotype, including, for example, resistance of cells expressing the polypeptide to a toxic agent such as neomycin.

The present invention also relates to a set of isogenic cell lines, which includes a first population of cells that are null for β-catenin expression, and at least a second population of cells that express a wild type β-catenin polypeptide that functions as an activated β-catenin polypeptide in the cells, wherein at least one of the populations of cells contains a disrupted β-catenin gene, and wherein, except for the a nucleotide sequence of the β-catenin gene, the first population of cells and the at least second population of cells are substantially genetically identical. In addition, the invention relates to a method of making such a set of isogenic cell lines by introducing a first recombinant nucleic acid molecule of the invention into parental cells that are homozygous for wild type β-catenin genes, wherein the β-catenin polypeptides expressed from the wild type β-catenin genes function as activated β-catenin polypeptides in the parental cells; selecting an intermediate population of cells, which is derived from a cell containing the recombinant nucleic acid molecule integrated into a wild type β-catenin gene; further selecting from the intermediate population of cells a first population of cells, which are derived from a cell containing a recombinant nucleic acid molecule of the invention integrated into each of the wild type β-catenin genes; and selecting at least a second population of cells, which is derived from a cell of the parental population of cells or a cell of the intermediate population of cells or both. The step of further selecting from the intermediate population of cells can be performed by determining whether a selected cell as having a disrupted wild type β-catenin gene also has the second wild type β-catenin gene disrupted; or can comprise introducing into the intermediate population of cells a second recombinant nucleic acid molecule of the invention, which can be the same or different from the first recombinant nucleic acid molecule, and selecting a second population of cells in which both wild type β-catenin genes are disrupted. The invention also relates to a set of isogenic human cell lines, which includes a first population of cells in which one or both β-catenin genes is disrupted, and at least a second population of cells that express a wild type β-catenin polypeptide, wherein, except for the a nucleotide sequence of the β-catenin gene, the first population of cells and the at least second population of cells are substantially genetically identical.

Accordingly, the present invention also provides sets of isogenic cell lines produced by the methods of the invention. A set of isogenic cell lines of the invention can include a first population of cells that express a wild type β-catenin polypeptide and at least a second population of cells that express an activated β-catenin polypeptide. In addition, the set can include at least a third population of cells that are null for β-catenin polypeptide expression; or that are heterozygous for the mutant β-catenin gene and the wild type β-catenin gene; or can include both such populations.

A set of isogenic cell lines of the invention also can include a first population of cells that are null for β-catenin expression, and at least a second population of cells that express a wild type β-catenin polypeptide that functions as an activated β-catenin polypeptide in the cells, wherein except for expression of the β-catenin polypeptide the cells are substantially genetically identical. The at least second population of cells can express a wild type β-catenin from one wild type β-catenin gene, wherein a second wild type β-catenin gene in the cell has been disrupted, or can express wild type β-catenin polypeptides from both of the wild type β-catenin genes. Such a set of isogenic cell lines also can include a first population of cells that are null for β-catenin expression, a second population of cells that are hemizygous for expression of a wild type β-catenin polypeptide that functions as an activated β-catenin polypeptide, and a third population of cells that is homozygous for expression of wild type β-catenin polypeptides that function as activated β-catenin polypeptides, i.e., cells have two, one or no disrupted wild type β-catenin genes.

The present invention also relates to a method of identifying a therapeutic agent that allows selective killing of cells expressing an activated β-catenin polypeptide, but not cells expressing a wild type β-catenin polypeptide. Such a screening assay of the invention can be performed, for example, by contacting an isogenic set of cells of the invention with a test agent to be examined for therapeutic activity, and identifying an agent that allows for the selective killing.

In one embodiment, the test agent is examined for the ability to selectively kill cells expressing an activated β-catenin polypeptide, for example, cancer cells. In this embodiment, the set of isogenic cells can be contacted with the test agent, and an agent that selectively kills those cells expressing the activated β-catenin polypeptide, but not those cells expressing the normal β-catenin polypeptide, can be identified. In another embodiment, the test agent is examined for the ability to protect cells that are expressing a normal β-catenin polypeptide, but not cells expressing an activated β-catenin polypeptide, from exposure to a modality that otherwise would be toxic to the cells. In this embodiment, the set of isogenic cells can be contacted with the test agent and the modality, and an agent that selectively protects those cells expressing the normal β-catenin polypeptide, but not those cells expressing the activated β-catenin polypeptide, can be identified.

A screening assay of the invention, which utilizes an isogenic set of cells that includes a populations of cell that express a wild type β-catenin and populations of cells that express an activated β-catenin polypeptide, and can include populations of cells that are heterozygous for wild type and activated β-catenin polypeptide expression or that are null for β-catenin expression, is particularly useful for screening a large number of test agents, for example, a combinatorial library of test agents. The test agent, which is to be examined for therapeutic activity, can be a peptide, a peptidomimetic, a polynucleotide, a small organic molecule, or any other agent. Where the agent is a polynucleotide, it generally is not a polynucleotide encoding a functional β-catenin polypeptide or a polynucleotide complementary thereto.

A therapeutic agent identified according to a method of the invention can act, for example, by correcting the defect in an activated β-catenin polypeptide that results in its unregulated activity, by killing cells that express the activated β-catenin polypeptide, by providing a selective advantage to cells that do not express the activated β-catenin polypeptide, or in any other way. Preferably, the therapeutic agent is toxic to cells that express an activated β-catenin polypeptide, but not to cells expressing a normal β-catenin polypeptide.

The present invention also relates to a method of treating a pathologic condition in a patient, wherein the pathologic condition is characterized, at least in part, by cells that express an activated β-catenin polypeptide. Such a method is performed by administering a therapeutic agent identified according to a method of the invention to the patient in an amount that, alone or in combination with one or more other modalities, provides a therapeutic advantage to the patient. A modality administered in combination with the therapeutic agent can facilitate the effect of the agent or can act independently of the agent. The pathologic condition can be any condition associated with cells that express activated β-catenin, particularly a cancer.

### BRIEF DESCRIPTION OF THE DRAWINGS

FIGS. 1A to 1F show the nucleotide sequence of a recombinant nucleic acid molecule containing a polynucleotide sequence encoding a neomycin resistance gene flanked by nucleotide sequences of the human β-catenin gene (SEQ ID NO: 1). A 5′ Hind III site and a 3′ Sal I site are shown in italics at each end of the sequence. The nucleotide sequence of the left homology arm of the β-catenin gene (nucleotides 7 to 6703) is shown beginning after the 5′ Hind III site and ending just before the initiator methionine ATG codon (ATG; nucleotides 6704 to 6706) for the neomycin resistance gene. The sequence of the neomycin resistance gene sequence nucleotide 6704 to 7574) is followed by a primer site used for PCR identification of β-catenin gene knockouts (underlined; nucleotides 7575 to 7594), which is then followed by the right homology arm of the β-catenin gene (nucleotides 7575 to 8844).

### DETAILED DESCRIPTION OF THE INVENTION

The present invention provides sets of isogenic cell lines. In one embodiment, the set includes a first population of cells that express a wild type β-catenin polypeptide, and at least a second population of cells that express an activated β-catenin polypeptide. A characteristic of a set of isogenic cell lines of the invention is that, except for the nucleotide sequence of the endogenous β-catenin genes, the first population of cells and the second (and other) population of cells are substantially genetically identical. As disclosed herein, such a set of isogenic cell lines can be obtained starting with a cell line, for example, a human cancer cell line, that is heterozygous for a wild type β-catenin gene and a mutant β-catenin gene that encodes an activated β-catenin polypeptide, independently knocking out one or the other of the β-catenin genes in cells of the cell line, and selecting populations of cells that contain only a wild type β-catenin gene or only a mutant β-catenin gene encoding an activate β-catenin polypeptide. In addition, the set can contain populations of cells that are heterozygous for β-catenin expression, i.e., the parental cell line; or cells that are null for β-catenin expression and are derived from the heterozygous cells but do not express either a wild type or an activated β-catenin polypeptide; or both populations of cells.

In another embodiment, the set includes a first population of cells that are null for β-catenin expression, and at least a second population of cells that express a wild type β-catenin polypeptide that functions as an activated β-catenin polypeptide in the cells, wherein at least one of the populations of cells contains a disrupted β-catenin gene, and wherein, except for the a nucleotide sequence of the β-catenin gene, the first population of cells and the at least second population of cells are substantially genetically identical. As disclosed herein, such a set of isogenic cell lines can be obtained starting with a cell line, for example, a human cancer cell line, that is homozygous for wild type β-catenin genes, wherein the expressed β-catenin polypeptides function as activated β-catenin polypeptides, knocking out one or both of the β-catenin genes in cells of the cell line, and selecting a first population of cells that has both wild type β-catenin genes disrupted and is null for β-catenin polypeptide expression, and at least a second population of cells that expresses a wild type β-catenin polypeptide that functions as an activated β-catenin, wherein the second population of cells is derived from the homozygous parental cell line or is derived from a cell of the parental cell line that has one of the wild type β-catenin genes disrupted.

In still another embodiment, the set includes a first population of human cells in which one or both β-catenin genes is disrupted, and at least a second population of human cells that express a wild type β-catenin polypeptide, wherein, except for the a nucleotide sequence of the β-catenin gene, the first population of cells and the at least second population of cells are substantially genetically identical. The human cells from which the set of isogenic cell lines is derived generally are cells that are involved in the etiology of a pathologic condition, including a condition in which aberrant regulation of β-catenin gene expression or of a gene regulated by β-catenin is involved in the etiology of the pathology.

Current methods of screening cancer cell lines for therapeutic agents are limited in that the panels of cell lines currently available lack appropriate control cell lines because the panels, such as that employed by the National Cancer Institute, are composed exclusively of cancer-derived cells. As such, the use of these cell lines makes it is difficult to distinguish between a compound that selectively kills cancer cells and compounds that kill all cells. As a result, an artificially high “hit rate” is obtained when potential therapeutic agents are examined using these panels of cell lines. The compositions and methods of the present invention obviate this limitation by providing sets of isogenic cell lines, wherein the cell lines of the sets are substantially genetically identical except for the presence of a wild type β-catenin gene or a mutant β-catenin gene encoding an activated β-catenin polypeptide.

As disclosed herein, except for the β-catenin gene, the cell lines comprising the set of isogenic cell lines otherwise are substantially genetically identical. As used herein, the term “substantially genetically identical,” when used in reference to cell lines comprising a set of isogenic cell lines, means that the genomes of the cell lines are the same except for minor mutations that can generally occur in cells as a result of normal cell division. The cell lines of a set are substantially identical as a result of their being derived from the same original cell population and their being treated identically during their selection and isolation. One skilled in the art readily can determine that cell lines of the set of isogenic cell lines are substantially genetically identical using routine methods, for example, examining the genomes for common restriction fragments following treatment with one or a few different restriction endonucleases, or by examining the genomes for common sequences in regions known to be characterized by the presence of single nucleotide polymorphisms.

In particular, the artisan will know to confirm that an introduced recombinant nucleic acid molecule used in the preparation of the cell lines of the set of isogenic cell lines of the invention is integrated into the same relative position in each of the cell lines, and that the recombinant nucleic acid molecule is present only once in each of the genomes or, if present in more than a single copy, is present in the same number and in the same position in the genomes of each of the cell lines of the set. Such a confirmatory examination of the cell lines can be facilitated by including, for example, nucleotide sequences useful as targets for PCR amplification of the introduced recombinant nucleic acid molecule, such that the integration sites in the cell lines readily can be determined, for example, by Southern blot analysis.

As used herein, the term “set of isogenic cell lines” refers to at least two separate populations of cells that are substantially genetically identical except for the nucleotide sequences of their β-catenin genes. For clarity, reference is made herein to a “first population of cells” or a “second population of cells” or the like. It should be recognized, however, that the use of the terms “first”, “second”, and the like is only to facilitate discussion of the different cell lines, and is not meant to suggest any particular order of the cell lines or any relative importance of the cell lines. Preferably, the cell lines of the set of isogenic cell lines are diploid (or haploid in the case of cell lines derived from a germ cell) or near diploid. As used herein, the term “near diploid” means that the cell lines, as compared to corresponding normal cells, have only one or a few additional chromosomes or chromosome fragments that are stably maintained in the cells through serial passage.

As used herein, the term “corresponding normal cell” refers the cell type, from which a set of isogenic cell lines of the invention are derived, as it generally exists in nature. Reference is made herein to a corresponding normal cell because, in general, a cell line of a set of isogenic cell lines of the invention that is derived, for example, from human colon adenocarcinoma cells that are heterozygous for a mutant β-catenin gene and selected as disclosed herein to be homozygous for the mutant β-catenin gene, can differ genotypically and phenotypically from normal colon cells.

As disclosed herein, a cell line of a set of isogenic cell lines of the invention can be homozygous for a wild type β-catenin gene or for a mutant β-catenin gene, which encodes an activated β-catenin polypeptide, or can be hemizygous for the β-catenin gene, and can further include a cell line that is null for β-catenin polypeptide expression. The terms “heterozygous” and “homozygous” are used herein to refer to the relationship of the β-catenin genes present on each chromosome pair of a diploid cell, specifically, as to whether the β-catenin genes are different and encode a wild type and an activated, β-catenin polypeptide, or are the same, respectively. As used herein, reference to cells that are “null” for β-catenin expression means that the cells produce neither a wild type nor an activated β-catenin polypeptide. In addition, the term “hemizygous” is used herein to refer to a cell that contains only a single functional β-catenin gene, which can encode either a wild type β-catenin polypeptide or an activated β-catenin polypeptide. It should be recognized that the term “hemizygous” is used herein to include cells that contain only a single β-catenin gene, as well as cells that contain only a single functional β-catenin gene, for example, a cell that contains both β-catenin genes (i.e., one on each chromosome pair of a diploid cell), but from only one of which a β-catenin polypeptide can be expressed. As disclosed herein, cells that are hemizygous and express only a wild type β-catenin polypeptide or only an activated β-catenin polypeptide were viable and were capable of being maintained in culture, apparently indefinitely. In addition, cells that are null for β-catenin expression can be viable and maintained in culture.

As used herein, the term “wild type,” when used in reference to a β-catenin gene, refers to the gene as it exists in nature in a normal organism, generally, a mammalian organism such as a human, including naturally occurring polymorphic forms of the gene. In comparison, the term “mutant,” when used in reference to a β-catenin gene, refers to a gene that encodes an activated β-catenin polypeptide. In addition, the term “activated β-catenin polypeptide” is used herein to refer to a β-catenin that is not subject to the control mechanisms that regulate the level of β-catenin expression in a normal cell. The presence of an activated β-catenin polypeptide in a cell can be identified, for example, by using an immunologic or chromatographic method to determine the amount of β-catenin in a cell, by determining that a β-catenin polypeptide lacks or has a mutation of a serine or threonine residue that, when phosphorylated, results in degradation of the polypeptide, or by determining the level of transcriptional activity of a reporter gene regulated by a TCF or LEF transcription factor (see below), and comparing the result to that obtained for a corresponding normal cell known to express a wild type β-catenin polypeptide. The β-catenin gene is an oncogene that can be activated, for example, due to mutations that affect the amino terminal regulatory region of the β-catenin polypeptide, including specific serine and threonine residues that otherwise can be phosphorylated to regulate β-catenin activity (Polakis, Genes Devel. 14:1837-1851, 2000, which is incorporated herein by reference). Activating mutations in the β-catenin gene were first identified in colon cancer and melanoma (Morin et al., Science 275:1787-1790, 1997; Rubinfeld et al., Science 275:1790-1792, 1997, each of which is incorporated herein by reference), and have since been described in a wide variety of tumor types, including malignant and non-malignant tumors (see Polakis, supra, 2000). β-catenin is involved in the WNT signaling pathway in vertebrates and the Wg signaling pathway in invertebrates. This pathway also includes the APC (adenomatous polyposis coli) gene product, which is a tumor suppressor that is absent or mutated in various cancers, including FAP. APC contains two β-catenin binding repeats, one of which is modulated by phosphorylation (Kinzler and Vogelstein, Cell 87:159-170, 1996, which is incorporated herein by reference). Mutations in APC are associated with increased levels of β-catenin, indicating that β-catenin is downstream of APC in the WNT/Wg signal transduction pathways. In addition, mutations in β-catenin that prevent its phosphorylation at specific serine or threonine residues are associated with elevated levels of β-catenin in cells. Elevated levels of β-catenin, in turn, are associated with increased transcriptional activity of genes regulated by TCF and LEF transcription factors, and also are associated with cell proliferative disorders, including tumor growth and cancers (Polakis, supra, 2000; Kinzler and Vogelstein, supra, 1996).

In one embodiment, a set of isogenic cell lines can be prepared from any cells that are heterozygous for a mutant β-catenin gene that encodes an activated β-catenin polypeptide, including a cell obtained from an organism containing cells heterozygous for a wild type and a mutant β-catenin gene, or cells that have been manipulated, for example, using a recombinant DNA method to contain a wild type and a mutant β-catenin gene. The cells can be obtained from an invertebrate or a vertebrate, and preferably are of mammalian origin, particularly of human origin. In one embodiment, the cells used to obtain a set of isogenic cells of the invention are a human cancer cell line, wherein the cancer cells are heterozygous with respect to the β-catenin gene and express an activated β-catenin polypeptide. Examples of such cells include HCT 116 human colon adenocarcinoma cells (ATCC CCL 247), HEC-1 A human endometrial cancer cells (ATCC HTB-112), and SW48 human colorectal adenocarcinoma cells (ATCC CCL-231).

In another embodiment, a set of isogenic cell lines can be prepared from any cell line that is homozygous for wild type β-catenin genes, wherein the β-catenin polypeptides expressed from the wild type β-catenin genes function as activated β-catenin polypeptides. As used herein, reference to a wild type β-catenin polypeptide that “functions as an activated β-catenin polypeptide” means that the wild type β-catenin polypeptide is refractory to regulation by the APC gene product or is refractory to targeted degradation via the ubiquitin pathway (see Polakis, supra, 2000). DLDI colon carcinoma cells (ATCC CCL-221) are an example of such cells, wherein the otherwise wild type β-catenin polypeptides function as an activated β-catenin due to a mutation of an APC tumor suppressor gene in the cells. As such, β-catenin in DLDI cells is refractory to regulation by APC, which normally is involved in regulating β-catenin signaling; the mutant APC gene product lacks this regulatory activity (see, for example, Polakis, supra, 2000). As such, DLDI cells, which are homozygous for wild type β-catenin genes and express a mutant APC gene product, have a phenotype similar to that of cells that express an activated β-catenin polypeptide.

The present invention also provides a set of isogenic transgenic non-human organisms, which are produced from a set of isogenic cell lines of the invention. The set of transgenic non-human organisms, which generally are vertebrate organisms, particularly mammals, include at least a first transgenic non-human organism, which is produced from a cell that expresses a wild type β-catenin polypeptide, and a second transgenic non-human organism, which is produced from a cell that expresses an activated β-catenin polypeptide, whereas the transgenic organisms in the set otherwise are substantially genetically identical.

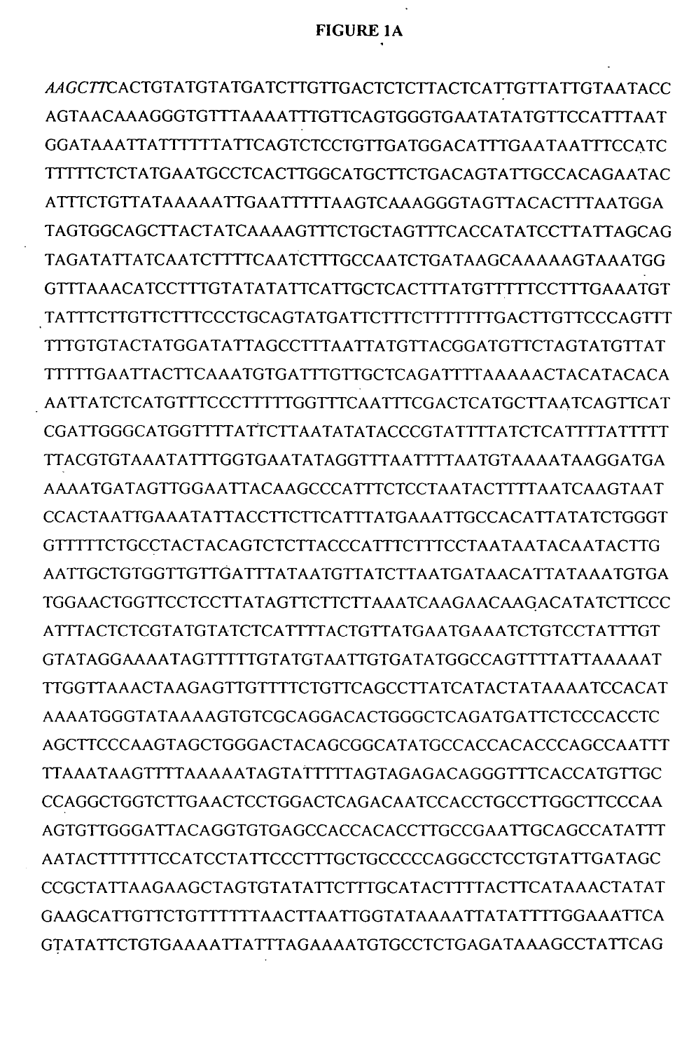

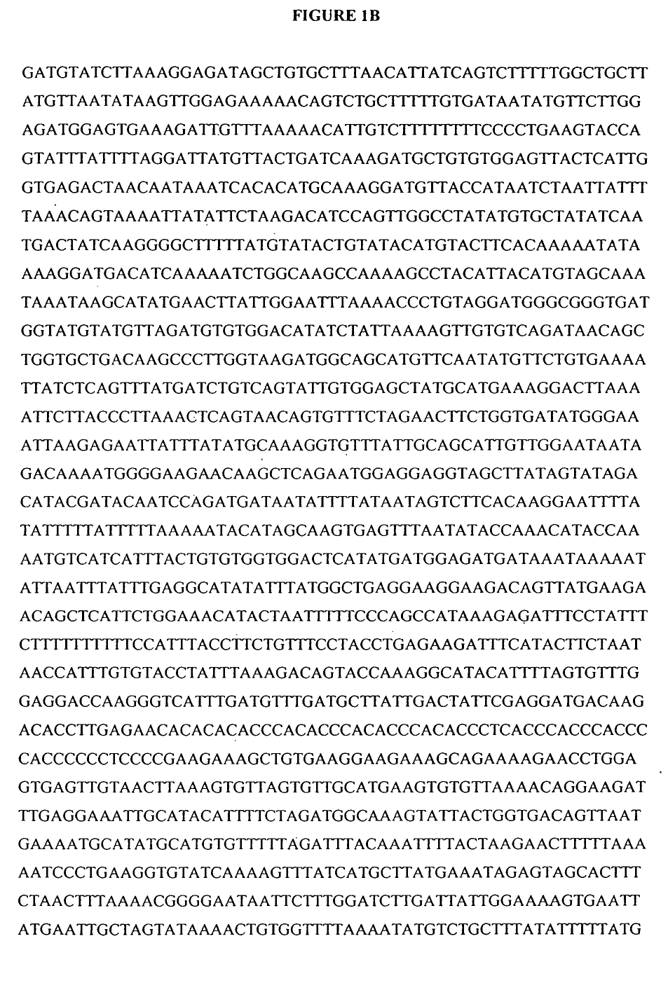

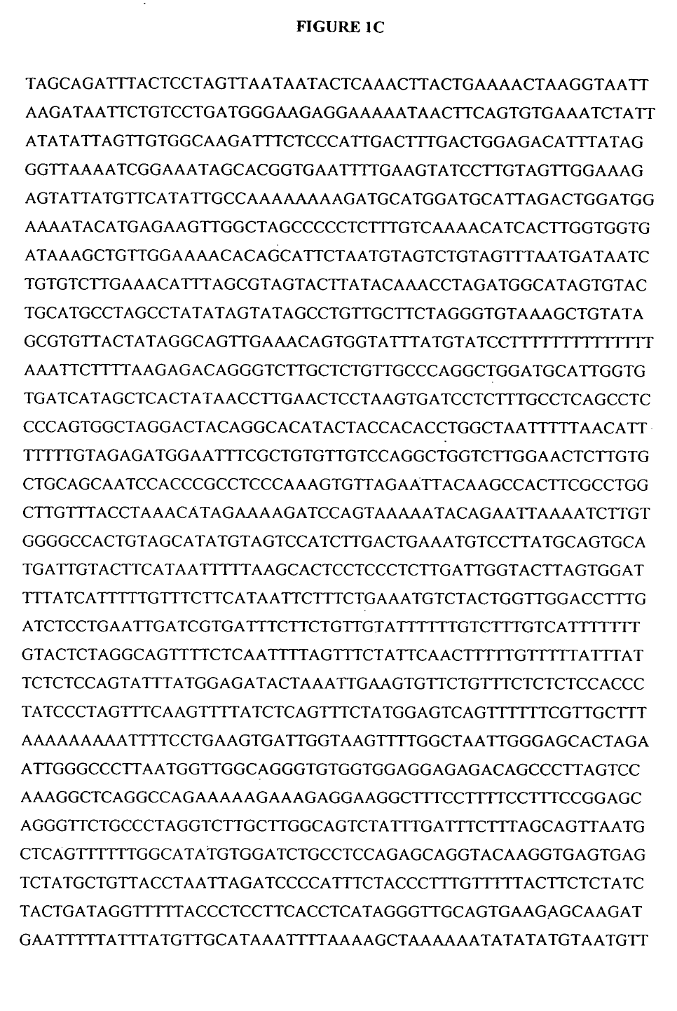

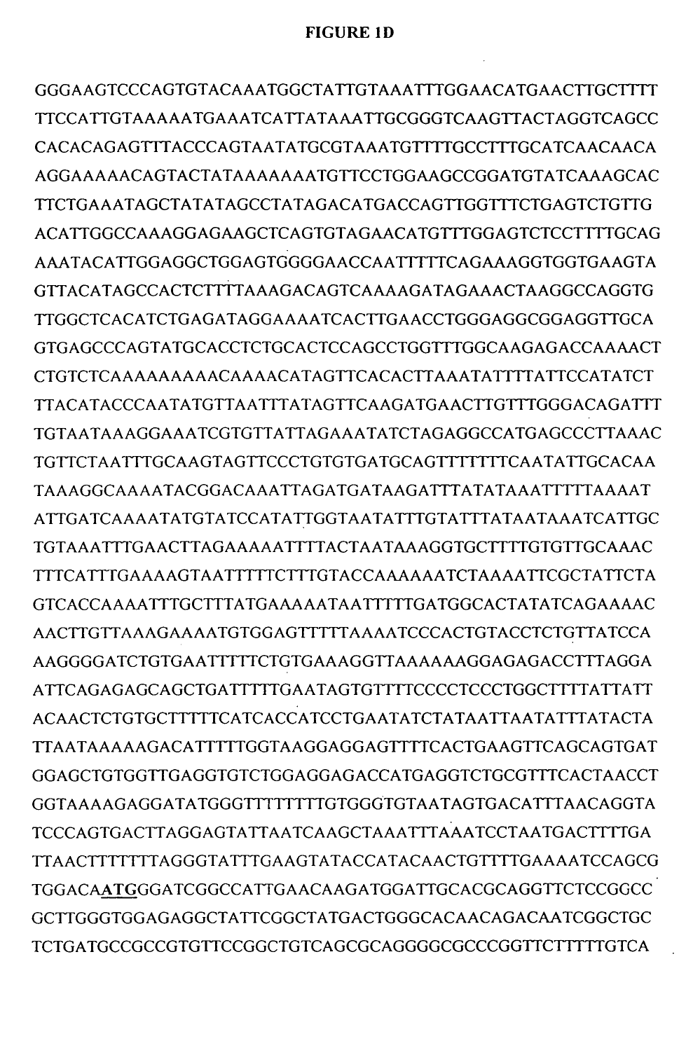

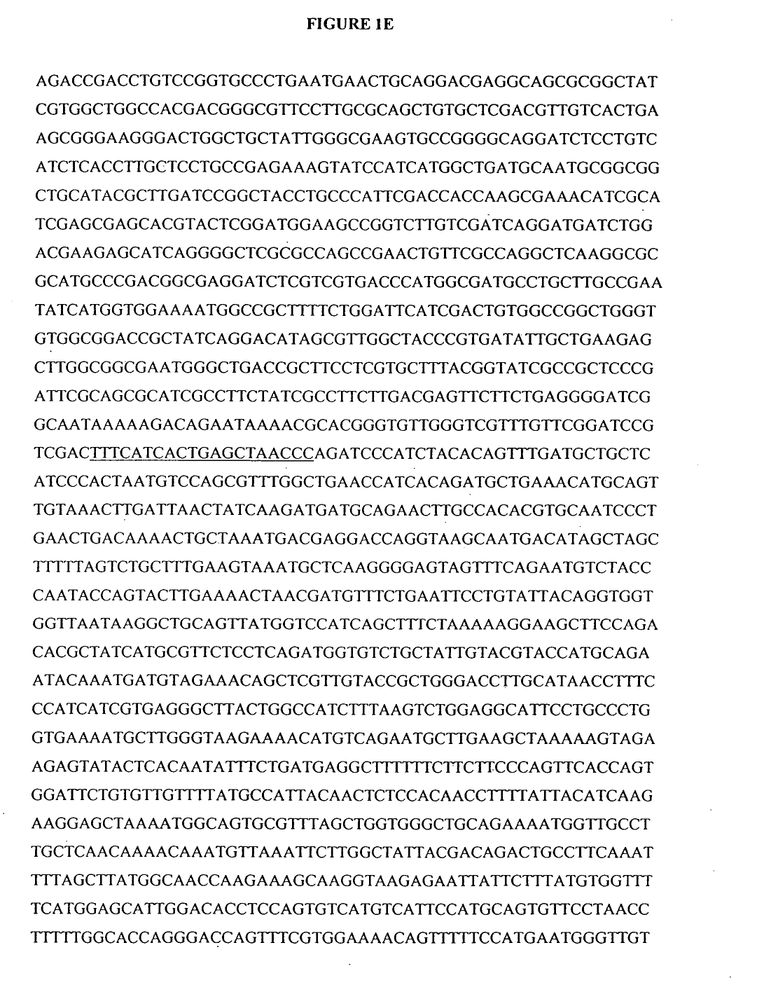

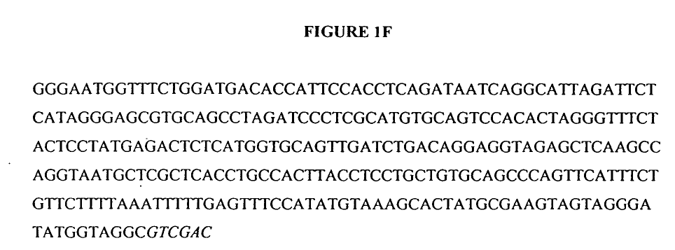

The invention also provides a recombinant nucleic acid molecule, which is composed of at least a first polynucleotide, which has a first end and a second end, i.e., is or can be linear, and is flanked at one end by a first nucleotide sequence of a β-catenin gene and at the second end by a second nucleotide sequence of a β-catenin gene. The first and second nucleotide sequences of the β-catenin gene are selected such that they can specifically hybridize to an endogenous β-catenin gene, including an endogenous wild type gene, a mutant gene, or both, in a cell. Generally, the first and second nucleotide sequences each are at least about 20 to 50 nucleotides in length, usually at least about 100 nucleotides in length, particularly about 500 to 1000 nucleotides in length, and can be several thousand nucleotides in length or more. A recombinant nucleic acid molecule of the invention is exemplified herein the nucleic acid molecule shown in FIG. 1, which includes a neomycin resistance gene flanked by sequences of the human β-catenin gene. As such, a recombinant nucleic acid molecule of the invention, which can be contained in a vector, is particularly useful for knocking out a wild type or mutant β-catenin gene in a cell by homologous recombination.

The flanking nucleotide sequences can be selected such that, upon integration into a cell genome, the polynucleotide contained within the recombinant nucleic acid molecule is operatively linked to the endogenous β-catenin transcriptional regulatory elements, translational regulatory elements, or both. The polynucleotide can contain all of the regulatory elements required for transcription of the polynucleotide and, if appropriate, translation of an encoded polypeptide, or can lack some or all of the transcriptional and translational regulatory elements. For example, the polynucleotide can encode a polypeptide such as an antibiotic resistance gene product, beginning with the amino acid immediately C-terminal to, but excluding, the initiator methionine sequence, and the first and second β-catenin gene nucleotide sequences can be selected such that the encoding polynucleotide, when integrated by homologous recombination, is positioned immediately C-terminal to the initiator methionine of the β-catenin gene (i.e., 5′ of the initiator methionine codon) such that the encoded polypeptide can be transcribed and translated in the cell.

The one or more polynucleotides contained in a recombinant nucleic acid molecule of the invention, generally are heterologous with respect to the target β-catenin gene. As used herein, the term “heterologous” refers to a polynucleotide that has a sequence that is different from an endogenous β-catenin gene, which can be a wild type or mutant gene. Generally, the polynucleotide is heterologous in that it is from a cell type other than the target cell type, which is the cell into which a recombinant nucleic acid molecule of the invention is to be introduced; or is from a different organism, which can be a prokaryotic or eukaryotic organism; or is a synthesized or recombinant polynucleotide that has a sequence different from that of the target endogenous β-catenin gene or genes. The heterologous polynucleotide generally encodes a polypeptide other than a wild type or mutant β-catenin polypeptide, and can be one of several operatively linked polynucleotides that encode a number of discrete polypeptides or a fusion polypeptide. However, the heterologous polynucleotide can be derived from a polynucleotide that encodes a β-catenin polypeptide, but that has been genetically modified such that, upon integration into an endogenous β-catenin gene, it disrupts transcription of the endogenous gene or translation of the encoded β-catenin polypeptide, for example, by introducing a frame shift or a STOP codon or the like into the endogenous gene such that the encoded endogenous β-catenin polypeptide is not expressed.

As used herein, the term “genetically modified” refers to the manipulation of a polynucleotide or of a cell such that the polynucleotide or cell is different from the naturally occurring polynucleotide or cell from which it is derived. Thus, as indicated above, a polynucleotide encoding a β-catenin polypeptide can be genetically modified, for example, by using a method such as site directed mutagenesis to change a codon encoding an amino acid of the β-catenin polypeptide into a STOP codon, such that the polypeptide is not expressed, or is expressed as a truncated inactive polypeptide. Similarly, such a polynucleotide can be genetically modified by deleting one or a few nucleotides such that a frame shift is introduced, thus resulting in the expression, if any, of an inactive or truncated β-catenin polypeptide. It will be recognized that such genetically modified polynucleotides can be heterologous with respect to an endogenous β-catenin gene, provided the sequence of the genetically modified polynucleotide is different from that of the corresponding sequence of an endogenous β-catenin gene.

A cell also can be genetically modified, for example, by introducing a heterologous polynucleotide or recombinant nucleic acid molecule into the cell such that the polynucleotide or nucleic acid molecule or a portion thereof is integrated into the genome of the cell. For example, a genetically modified cell can be a cell that has been treated according to a method of the invention such that both endogenous β-catenin genes have been knocked out, and has been further manipulated by introducing a polynucleotide encoding a wild type or an activated β-catenin polypeptide into the cell.

As used herein, the term “operatively linked” refers that two or more molecules, particularly polynucleotides or polypeptides, that are positioned with respect to each other such that they act as a single unit and effect a function attributable to one or both molecules or a combination thereof. For example, a polynucleotide sequence encoding an antibiotic resistance gene product can be operatively linked to a transcriptional regulatory element, in which case the regulatory element confers its regulatory effect on the polynucleotide similarly to the way in which the regulatory element would effect a polynucleotide sequence with which it normally is associated with in a cell. A first polynucleotide coding sequence also can be operatively linked to a second (or more) coding sequence such that a fusion polypeptide, in which the two (or more) encoded polypeptides are translated into a single polypeptide, i.e., are covalently bound through a peptide bond, can be produced.

A polypeptide expressed from a polynucleotide of a recombinant nucleic acid molecule of the invention can confer a detectable phenotype on the cell. As used herein, the term “detectable phenotype” means that the expressed polypeptide provides a means to identify or select a cell containing the recombinant nucleic acid molecule. The detectable phenotype can be, for example, an increased resistance or susceptibility of a cell expressing the polypeptide to a toxic agent, such as increased resistance to an antibiotic, or can be a production of signal such as a fluorescent, luminescent, chemiluminescent or the like signal. Such an encoded or expressed polypeptide also is referred to herein as a “reporter molecule” or a “selectable marker.”

Reporter molecules, which confer a detectable phenotype on a cell, are well known in the art and include, for example, fluorescent polypeptides such as green fluorescent protein, cyan fluorescent protein, red fluorescent protein, or enhanced forms thereof, an antibiotic resistance polypeptide such as puromycin N-acetyltransferase, hygromycin B phosphotransferase, neomycin (aminoglycoside) phosphotransferase, and the Sh ble gene product; a cell surface protein marker such as the cell surface protein marker neural cell adhesion molecule (N-CAM); an enzyme such as a β-lactamase, chloramphenicol acetyltransferase, adenosine deaminase, aminoglycoside phosphotransferase, dihydrofolate reductase, thymidine kinase, luciferase or xanthine guanine phosphoribosyltransferase polypeptide; or a peptide tag such as a c-myc peptide, a polyhistidine, a FLAG epitope, or any ligand (or cognate receptor), including any peptide epitope (or antibody, or antigen binding fragment thereof, that specifically binds the epitope; see, for example, Hopp et al., BioTechnology 6:1204 (1988); U.S. Pat. No. 5,011,912, each of which is incorporated herein by reference). Expression of a reporter molecule can be detected using the appropriate instrumentation or reagent, for example, by detecting fluorescence of a green fluorescent protein or light emission upon addition of luciferin to a luciferase reporter molecule, or by detecting binding of nickel ion to a polypeptide containing a polyhistidine tag. Similarly, expression of a selectable marker such as an antibiotic can be detected by identifying the presence of cells growing under the selective conditions.

A reporter molecule also can provide a means of isolating or selecting a cell expressing the reporter molecule. For example, the reporter molecule can be a polypeptide that is expressed on a cell surface and that contains an operatively linked c-myc epitope; an anti-c-myc epitope antibody can be immobilized on a solid matrix; and cells, some of which express the tagged polypeptide, can be contacted with the matrix under conditions that allow selective binding of the antibody to the epitope. Unbound cells can be removed by washing the matrix, and bound cells, which express the reporter molecule, can be eluted and collected. Methods for detecting such reporter molecules and for isolating the molecules, or cells expressing the molecules, are well known to those in the art (see, for example, Hopp et al., supra, 1988; U.S. Pat. No. 5,011,912). As indicated above, a convenient means of isolating and selecting cells expressing a reporter molecule is provided by using a reporter molecule that confers antibiotic resistance, and isolating cells that grow in the presence of the particular antibiotic.

Where the polynucleotide contained in a recombinant nucleic acid molecule comprises two or more operatively linked polynucleotides, one polynucleotide can encode a selectable marker, and additional polynucleotides can be complementary to PCR primers, which can be used to amplify a portion of the recombinant nucleic acid molecule. Two or more operatively linked polynucleotides also can encode two or more selectable markers, for example, an antibiotic resistance gene product, which can be used as a first selection for cells containing the integrated recombinant nucleic acid molecule, and a green fluorescent protein, which can be used as a second selection method such as a fluorescence activated cell sorting method. A variety of combinations of polynucleotides can be used in a recombinant nucleic acid molecule of the invention, and can be selected based, for example, based on availability, convenience of use, and the like.

The present invention also provides a vector containing a recombinant nucleic acid molecule of the invention. Such a vector, which can be referred to as a targeting vector, can facilitate manipulation of the recombinant nucleic acid molecule and introduction of the recombinant nucleic acid molecule into a target cell. A vector generally contains elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCOIBRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J Bioenerg. Biomemb. 25:37-42, 1993; Kirshenbaum et al.,J Clin. Invest. 92:381-387, 1993; each of which is incorporated herein by reference).

A vector also can contain a selectable marker, which is independent of one that may be encoded by a recombinant nucleic acid molecule of the invention, such that a host cell, for example, containing the vector can be selected, and can include a promoter sequence, which can provide constitutive, inducible, or tissue specific expression of the selectable marker, thus providing a means to select a particular cell type, for example, from among a mixed population of cells containing the introduced vector and recombinant nucleic acid molecule contained therein. The vector can be a cloning vector or an expression vector, and can be a plasmid or a viral vector, the latter of which can be selected based on its ability to infect one or few specific cell types with relatively high efficiency. For example, the viral vector can be derived from a virus that infects particular cells of an organism of interest, for example, vertebrate host cells such as mammalian host cells. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980-990, 1992; Anderson et al., Nature 392:25-30 Suppl.,1998; Verma and Somia, Nature 389:239-242, 1997; Wilson, New Engl. J Med. 334:1185-1187 (1996), each of which is incorporated herein by reference).

When retroviruses, for example, are used for gene transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

A recombinant nucleic acid molecule of the invention, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

Introduction of a recombinant nucleic acid molecule into a cell by infection with a viral vector is particularly advantageous in that it can efficiently introduce the recombinant nucleic acid molecule into a cell ex vivo or in vivo (see, for example, U.S. Pat. No. 5,399,346, which is incorporated herein by reference). Moreover, viruses are very specialized and can be selected as vectors based on an ability to infect and propagate in one or a few specific cell types. Thus, their natural specificity can be used to target the nucleic acid molecule contained in the vector to specific cell types. As such, a vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events. Accordingly, the present invention also provides a host cell containing a recombinant nucleic acid molecule of the invention, which can be contained in a vector.

The present invention also provides a method of producing a set of isogenic cell lines, which includes a first population of cells that express a wild type β-catenin polypeptide and at least a second population of cells that express an activated β-catenin polypeptide, wherein the isogenic cell lines are substantially genetically identical except with respect to the β-catenin gene encoding the β-catenin polypeptide. In one embodiment, the method is performed by introducing a recombinant nucleic molecule, which comprises a polynucleotide flanked by nucleotide sequences of an endogenous β-catenin gene as disclosed herein, into cells that are heterozygous for a mutant β-catenin gene, which encodes an activated β-catenin polypeptide, and a wild type β-catenin gene, which encodes a wild type β-catenin polypeptide; and selecting a first population of cells, which are derived from a cell containing the recombinant nucleic acid molecule integrated into only the mutant β-catenin gene and expressing only the wild type β-catenin polypeptide; and at least a second population of cells, which are derived from a cell containing the recombinant nucleic acid molecule integrated into only the wild type β-catenin gene and expressing only the activated β-catenin polypeptide. If desired, the method can further include selecting at least a third population of cells, which are derived from a cell containing the recombinant nucleic acid molecule integrated into both the mutant β-catenin gene and the wild type β-catenin gene, wherein said cells are null for β-catenin polypeptide expression.

In another embodiment, the method is performed by introducing a recombinant nucleic molecule, which comprises a polynucleotide flanked by nucleotide sequences of an endogenous β-catenin gene as disclosed herein, into cells that are heterozygous for a mutant β-catenin gene, which encodes an activated β-catenin polypeptide, and a wild type β-catenin gene, which encodes a wild type β-catenin polypeptide; selecting a first population of cells, which are derived from a cell containing the recombinant nucleic acid molecule integrated into both endogenous β-catenin genes and are null for β-catenin polypeptide expression; and at least a second population of cells, which are derived from a cell containing the recombinant nucleic acid molecule integrated either into the wild type β-catenin gene and express only the activated β-catenin polypeptide, or into the mutant β-catenin gene and express only the wild type β-catenin polypeptide; and genetically modifying the first population of cells by introducing into the cells a polynucleotide encoding a wild type β-catenin polypeptide, where the second population of cells expresses an activated β-catenin polypeptide, or a polynucleotide encoding an activated β-catenin polypeptide, where the second population of cells expresses a wild type β-catenin polypeptide. If desired, a portion of the first population of cells can be maintained without genetic modification, thereby providing a population of cells that are null for β-catenin polypeptide expression.

The cells that are heterozygous for the mutant and wild type β-catenin genes and used to produce a set of isogenic cell lines according to a method of the invention can be derived from any organism that expresses a β-catenin polypeptide and from any cell type in the organism. Generally, the cells are obtained from a vertebrate, particularly a mammal. Preferably the cells are human cells that are known to express or have a propensity to express an activated β-catenin polypeptide, particularly wherein such expression is associated with a pathological condition, including any of a variety of malignant and non-malignant tumors such as colon cancer and melanoma, desmoid tumors, which are common in patients with FAP, hepatoblastoma, medulloblastoma, thyroid cancers, hepatocellular carcinoma, Wilm's tumor, and prostate cancer (see, for example, Morin et al., supra, 1997; Rubinfeld et al., supra, 1997; Polakis, supra, 2000). Thus, in one embodiment, the cells that are used in a method of the invention are human cells obtained from such a malignant or non-malignant tumor, for example, human cancer cells such a HCT116 human colon adenocarcinoma cell lines.

According to a method of the invention, the recombinant nucleic acid molecule can be integrated into the cell genome by homologous recombination into an endogenous β-catenin gene, wherein the β-catenin nucleotide sequences flanking the polynucleotide in the recombinant nucleic acid molecule target the construct into the endogenous gene. When the polynucleotide in the recombinant nucleic acid molecule encodes a polypeptide, the β-catenin nucleotide sequences in the recombinant nucleic acid molecule can be selected such that, upon integration in the β-catenin gene in the genome, the polypeptide is expressed from the transcription or translation regulatory elements or both of the endogenous gene, thereby conferring a detectable phenotype on the cell due to expression of the polypeptide. As disclosed herein, the detectable phenotype can be any phenotype, including, for example, resistance of cells expressing the polypeptide to a toxic agent such as neomycin acetyltransferase or other antibiotic, thereby allowing the selection of cell lines as desired and production of a set of isogenic cell lines.

Accordingly, the present invention also provides a set of isogenic cell lines produced by a method of the invention. Such a set of isogenic cell lines can include a first population of cells that express a wild type β-catenin polypeptide and at least a second population of cells that express an activated β-catenin polypeptide. In addition, the set can include at least a third population of cells that are null for β-catenin polypeptide expression; or that are heterozygous for the mutant β-catenin gene and the wild type β-catenin gene; or can include both such populations.

In addition, the invention provides a method of making a set of isogenic cell lines, including a first population of cells that are null for β-catenin expression, and at least a second population of cells that express a wild type β-catenin polypeptide that functions as an activated β-catenin polypeptide in the cells. Such a method can be performed, for example, by introducing a first recombinant nucleic acid molecule of the invention into parental cells that are homozygous for wild type β-catenin genes, wherein the β-catenin polypeptides expressed from the wild type β-catenin genes function as activated β-catenin polypeptides in the parental cells; selecting an intermediate population of cells, which is derived from a cell containing the recombinant nucleic acid molecule integrated into a wild type β-catenin gene; further selecting from the intermediate population of cells a first population of cells, which are derived from a cell containing a recombinant nucleic acid molecule of the invention integrated into each of the wild type β-catenin genes; and selecting at least a second population of cells, which is derived from a cell of the parental population of cells or a cell of the intermediate population of cells or both.

Generally, the cells of the intermediate population of cells contains a single disrupted β-catenin gene due to integration of the first recombinant nucleic acid molecule. As disclosed herein, such cells can comprise a population of cells of an isogenic set of cells of the invention, thus providing a population of cells that is hemizygous for a wild type β-catenin gene and that expresses a wild type β-catenin polypeptide that functions as an activated β-catenin. In addition, cells of the intermediate population of cells are used to obtain a first population of cells of the isogenic set, wherein the first population of cells are null for β-cateriin expression. The first population of cells can be obtained by selecting cells of the intermediate population that have both β-catenin genes disrupted due to integration of the first recombinant nucleic acid molecule into both wild type β-catenin genes. Alternatively, a second recombinant nucleic acid molecule of the invention, which can encode a different selectable marker, for example, can be introduced into cells of the intermediate population of cells that contain a single disrupted β-catenin gene, and cells having the remaining wild type β-catenin gene disrupted can be selected, such that a first population of cells of the isogenic set can be derived.

Accordingly, the present invention also provides a set of isogenic cell lines produced such a method. Such a set of isogenic cell lines include a first population of cells that are null for β-catenin expression, and at least a second population of cells that express a wild type β-catenin polypeptide that functions as an activated β-catenin polypeptide in the cells, wherein the cells of the at least second population can express a wild type β-catenin from one wild type β-catenin gene, wherein the second wild type β-catenin gene in the cell has been disrupted, or can express wild type β-catenin polypeptides from both of the wild type β-catenin genes. The set of isogenic cell lines also can include a first population of cells that has both wild type β-catenin genes disrupted and are null for β-catenin expression, a second population of cells that have one wild type β-catenin gene disrupted and are hemizygous for expression of a wild type β-catenin polypeptide that functions as an activated β-catenin polypeptide, and a third population of cells that is homozygous for expression of wild type β-catenin polypeptides that function as activated β-catenin polypeptides, i.e., the parental cells.

The present invention also provides a method of using a set of isogenic cell lines to identify a therapeutic agent that allows selective killing of cells expressing an activated β-catenin polypeptide. As used herein, reference to “selective killing of cells expressing an activated β-catenin polypeptide” means that such cells are more susceptible to the effect of a toxic agent than are cells that express a wild type β-catenin polypeptide but otherwise are substantially genetically identical to the cells expressing the activated β-catenin polypeptide. As disclosed herein, such selective killing can be due to killing by a therapeutic agent identified according to a method of the invention, or can be due to the ability of the therapeutic agent to protect the cells expressing the wild type β-catenin polypeptide to a toxic agent to a greater extent than the therapeutic agent protects the cells expressing the activated β-catenin polypeptide. Methods for determining whether a therapeutic agent allows for selective killing as desired are well known and include, for example, determining a dose or concentration of an agent that produces a percentage greater killing of the cells expressing the activated β-catenin polypeptide as compared to cells expressing the wild type β-catenin polypeptide such as 10% greater killing, or 50% greater killing, or 90% greater killing, or the like. Killing of cells can be examined, for example, using a vital stain such as trypan blue, which accumulates in dead cells, using a DNA binding dye such as propidium iodide or H33258 and examining the genomic DNA for a fragmentation characteristic of apoptosis, or measuring the incorporation of a labeled nucleoside such as tritiated thymidine or a nucleoside analog such as bromodeoxyuridine into the cellular DNA (see, for example, U.S. Pat. No. 5,897,999, which is incorporated herein by reference).

A method of identifying a therapeutic agent can be performed, for example, by contacting the cell populations of an isogenic set of cell lines of the invention, independently or together, with at least a test agent to be examined as a potential therapeutic agent, and detecting selective killing of cells expressing the activated β-catenin polypeptide as compared to cells expressing the wild type β-catenin polypeptide. Where the test agent is being examined for an ability to selectively kill the cells expressing the activated β-catenin polypeptide, there is no requirement that any agent other than the test agent be contacted with the cells of the isogenic set. In comparison, where the test agent is being examined for an ability to protect cells expressing a wild type β-catenin polypeptide from a toxic agent, the method further includes contacting the set of isogenic cell lines with the toxic agent, for example, a cancer chemotherapeutic agent where the cells expressing the activated β-catenin polypeptide are derived from cancer cells such as human cancer cells.

The term “test agent” is used herein to mean any agent that is being examined for agonist or antagonist activity in a method of the invention. Although the method generally is used as a screening assay to identify previously unknown molecules that can act as a therapeutic agent that allows for selective killing of cells expressing an activated β-catenin polypeptide, a method of the invention also can be used to confirm that an agent known to have such activity, in fact has the activity, for example, in standardizing the activity of the therapeutic agent.

A test agent can be any type of molecule, including, for example, a peptide, a peptidomimetic, a polynucleotide, or a small organic molecule, that one wishes to examine for the ability to act as a therapeutic agent, which is an agent that provides a therapeutic advantage to a subject receiving it. It will be recognized that a method of the invention is readily adaptable to a high throughput format and, therefore, the method is convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res., 285:99-128, 1996; Lianget al., Science, 274:1520-1522, 1996; Dinget al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, BioTechnology, 13:351-360, 1995; each of which is incorporated herein by reference). Polynucleotides can be particularly useful as agents that can modulate a specific interaction of myostatin and its receptor because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference). Accordingly, the present invention also provides a therapeutic agent identified by such a method, for example, a cancer therapeutic agent.

Also provided is a method of identifying a therapeutic agent that allows selective killing of cells expressing an activated β-catenin polypeptide by administering a test agent to an experimental animal containing localized deposits of one or more populations of cells of a set of isogenic cell lines. The populations of cells comprising the set all can be present in the same animal, generally at independent and different locations. Preferably, at the population of cells expressing the wild type β-catenin polypeptide and the population of cells expressing the activated β-catenin polypeptide are contained in the same animal, thereby providing internal standardization as to the ability of an agent to allow selective killing. Depending on the type of therapeutic agent, i.e., whether the agent directly kills the cells or whether it protects cells from an otherwise toxic agent, the test agent can, but need not be, administered to the animal alone, or will be administered with the toxic agent, respectively.

The experimental animal generally is selected as one that is an immunodeficient animal, particularly where the cells of the isogenic set of cell lines is heterologous to the animal, for example, where the set of isogenic cells is produced from human cells such as human cancer cells. In addition, the experimental animal can be one of an inbred strain of animals such that the individual populations of cells of an isogenic set can be deposited into different animal with less concern that individual genetic differences among the animals will influence the results observed during the screening. Inbred animals and immunodeficient animals are well known and readily available from non-profit organizations and commercial sources such as the Jackson Laboratory, Charles River Laboratory, and the like.

The invention further provides a method of treating a cancer patient having a cancer characterized, in part, by cancer cells that express an activated β-catenin polypeptide. Such a method can be practiced, for example, by administering to the patient a cancer therapeutic agent identified according to a method of the invention in an amount sufficient to allow selective killing of the cancer cells in the patient. Where the therapeutic agent is one that protects cells expressing a wild type β-catenin polypeptide from an otherwise toxic agent, for example, a chemotherapeutic agent, a radiotherapeutic treatment, or the like, the subject also will be treated by administration of that agent, as well as with any other agent or combination of agents generally used to treat a patient having the particular type of cancer.

An amount of a therapeutic agent sufficient to allow the selective killing of cancer cells in a subject can be determined using routine clinical methods, including Phase I, II and III clinical trials. Efficacy of the method similarly can be determined using routine clinical methods, which will depend in part on the particular type of cancer, and include monitoring the clinical signs and symptoms known to be attributable to the cancer and indicative of the status of the disease.

A convenient pre-clinical method of determining efficacy of a therapeutic agent utilizes examination of the agent in an experimental animal containing localized deposits of one or more populations of cells of a set of isogenic cell lines. Preferably, the animal contains, at independent locations, at least the population of cells expressing the wild type β-catenin polypeptide and the population of cells expressing the activated β-catenin polypeptide, thereby providing internal standardization of the determination of efficacy of the therapeutic agent to allow selective killing. However, where the animal is one of an inbred strain of animals, the individual populations of cells of an isogenic set can be in different animals.

A therapeutic agent identified according to a method of the invention generally is administered to a living subject as a composition that includes a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the therapeutic agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. It will be recognize that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second agent such as a toxic agent, for example, a cancer chemotherapeutic agent, particularly where the therapeutic agent is one that protects cells expressing a wild type β-catenin polypeptide from the effect of the toxic agent.

The therapeutic agent can be incorporated within an encapsulating material such as into an oil-in-water emulsion, a microemulsion, micelle, mixed micelle, lipo some, microsphere or other polymer matrix (see, for example, Gregoriadis, Liposome Technology, Vol. 1 (CRC Press, Boca Raton, Fla. 1984); Fraley, et al., Trends Biochern. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J Clin. Invest., 91:2580-2585 (1993), which is incorporated herein by reference). In addition, a polynucleotide agent can be introduced into a cell using, for example, adenovirus-polylysine DNA complexes (see, for example, Michael et al., J Biol. Chem. 268:6866-6869 (1993), which is incorporated herein by reference).

The route of administration of a pharmaceutical composition containing the therapeutic agent will depend, in part, on the chemical structure of the therapeutic agent. Peptides and polynucleotides, for example, are not particularly useful when administered orally because they can be degraded in the digestive tract. However, methods for chemically modifying peptides, for example, to render them less susceptible to degradation by endogenous proteases or more absorbable through the alimentary tract are well known (see, for example, Blondelle et al., supra, 1995; Ecker and Crooke, supra, 1995). In addition, a peptide agent can be prepared using D-amino acids, or can contain one or more domains based on peptidomimetics, which are organic molecules that mimic the structure of peptide domain; or based on a peptoid such as a vinylogous peptoid.

A pharmaceutical composition as disclosed herein can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. A pharmaceutical composition also can be administered to the site of a pathologic condition, for example, intravenously or intra-arterially into a blood vessel supplying a tumor.

The total amount of a therapeutic agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the pharmaceutical composition to treat a pathologic condition in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration also are determined, initially, using Phase I and Phase II clinical trials.

The pharmaceutical composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The following example is intended to illustrate but not limit the invention.

### EXAMPLE 1

### Preparation and Characterization of a Set of Isogenic Cell Lines for β-Catenin

This example provides a method of producing a set of isogenic cell lines, including a population of cells expressing a wild type β-catenin polypeptide and a population of cells expressing an activated β-catenin polypeptide.

A PCR product derived from exon II of the human β-catenin gene was used to screen a human genomic BAC library. Four BACs were identified, prepared, and mapped using Southern blot analysis to identify fragments suitable for subcloning. The identified fragments were subcloned and sequenced, and a 9.8 kb contig of the human β-catenin genomic sequence containing exons 2 to 6 was generated. The subcloned fragments were used to create a promoterless targeting vector (see Waldman et al., Cancer Res. 55:5187-5190, 1995, which is incorporated herein by reference; see, also, U.S. Pat. No. 5,897,999).

The targeting vector was generated by subcloning Hind III/Sal I restriction fragments of the human β-genomic DNA sequence into the Hind III/Xho I site of the yeast shuttle vector, pRS425 (GenBank Accession No. U03452). Homologous recombination in yeast was then employed to replace exons of β-catenin with a promoterless neo^{R}gene. Briefly, PCR was used to generate an engineered neo^{R}gene with 40 nucleotide “tails” of β-catenin homology. The primers were designed to create a β-catenin/ neo^{R}gene junction, in which the initiating methionine of β-catenin was precisely replaced by the initiating methionine of the neo^{R}gene (see FIG. 1; neomycin ATG codon in bold and underlined).

In addition, a PCR priming site was engineered just 3′ to the neo^{R}gene, and 5′ to the right homology aim of the vector (see FIG. 1; primer underlined). The sequence of this priming site is identical to a sequence present in the β-catenin genomic region that is deleted upon successful homologous integration of the targeting vector into the human genome. Use of a PCR primer with a sequence identical to this priming site together with a primer complementary to sequences downstream (3′ ) to the right homology arm allows the unambiguous identification of β-catenin gene knockouts by PCR. The knock outs generally are confinned by Southern blot analysis. In cells in which the targeting vector has integrated randomly, the size of the PCR product of specific will be different from that obtained from cells in which the targeting vector has integrated via homologous recombination, the latter being of a predictable size depending the sequence of the 3′ primer.

The PCR product was co-transformed into S. cerevisiae with the linearized yeast shuttle vector. Yeast colonies were obtained and screened by PCR to identify colonies in which homologous recombination between the vector and the PCR product occurred. This completed β-catenin targeting vector then was shuttled into E. coli, amplified, and the sequence was determined to confirm the integrity of the junctions and the neo^{R}gene (see FIG. 1).

The promoterless human β-catenin targeting vector was linearized in the vector backbone and transfected into human HCT 116 cells, which are derived from a colon adenocarcinoma, are diploid, and contain one wild type and one mutant, oncogenic allele of β-catenin. Geneticin^{R}antibiotic resistant clones were obtained by limiting dilution, and 187 colonies were obtained. Genomic DNA was prepared and screened by PCR. 18% of the clones were knock outs (all were confirmed by Southern blot analysis) demonstrating that the targeting vector worked at high efficiency and can be generally useful for targeting β-catenin in a wide variety of human cells, including human cancer cells.

PCR sequencing was used to examine the remaining non-targeted allele. In 35% of the targeted clones, the remaining allele was wild type, and in 65% of the clones the remaining allele was mutant. This result demonstrates that the oncogenic form of β-catenin is not required for in vitro growth of the colon cancer cells.

Using these methods, human DLDI colon carcinoma cells have been targeted and a cell line containing one copy of the wild type β-catenin gene deleted has been obtained.

Although the invention has been described with reference to the above examples, it will be understood that modifications and variations are encompassed within the spirit and scope of the invention. Accordingly, the invention is limited only by the following claims.

1. A set of isogenic cell lines, comprising:
a first population of cells, which express only a wild type β-catenin polypeptide; and
at least a second population of cells, which express only an activated β-catenin polypeptide,
wherein at least one of the first population of cells or the at least second population of cells contains a disrupted β-catenin gene, and
wherein, except for a nucleotide sequence of a β-catenin gene, the first population of cells and the at least second population of cells are substantially genetically identical.

2. (canceled)

3. The set of isogenic cell lines of claim 1, wherein the cells of the first population of cells and the at least second population of cells are diploid.

4. The set of isogenic cell lines of claim 1, wherein the cells of the first population of cells are hemizygous for a wild type β-catenin gene.

5-7. (canceled)

8. The set of isogenic cell lines of claim 1, further comprising at least a third population of cells, wherein the third population of cells. when present, optionally.
a) expresses a wild type β-catenin polypeptide and an activated β-catenin polypeptide.
b) is heterozygous for a wild type β-catenin gene and a mutant β-catenin gene, which encodes the activated β-catenin polypeptide;
c) is hemizygous for a wild type β-catenin gene, and has been genetically modified to contain a polynucleotide encoding an activated β-catenin polypeptide: or
d) is null for β-catenin expression.

9-12. (canceled)

13. The set of isogenic cell lines of claim 1, wherein the cells of the first population of cells and the cells of the second population cells are mammalian cells.

14. The set of isogenic cell lines of claim 13, wherein the mammalian cells are human cells.

15. The set of isogenic cell lines of claim 14, wherein the human cells are derived from human cancer cells.

16. The set of isogenic cell lines of claim 15, wherein the human cancer cells are HCT116 human colon adenocarcinoma cell lines.

17. The set of isogenic cell lines of claim 1, comprising:
a) a first population of cells, which express a wild type β-catenin polypeptide, wherein said first population of cells is hemizygous for a wild type β-catenin gene;
a second population of cells, which express an activated β-catenin polypeptide, wherein the second population of cells is hemizygous for a mutant β-catenin gene, which encodes the activated β-catenin polypeptide; and
a third population of cells, which is null for β-catenin expressions optionally, further comprising
b) at least a fourth population of cells, which expresses a wild type β-catenin polypeptide, wherein said at least fourth population of cells is homozygous for a wild type β-catenin gene, or
at least a fourth population of cells, which expresses an activated β-catenin polypeptide, wherein said at least fourth population of cells is homozygous for a mutant β-catenin gene, which encodes the activated β-catenin polypeptide: or
c) a fourth population of cells. which expresses a wild type β-catenin polypeptide, wherein said fourth population of cells is homozygous for a wild type β-catenin gene; and
at least a fifth population of cells, which expresses an activated β-catenin polypeptide, wherein said at least fifth population of cells is homozygous for a mutant β-catenin gene, which encodes the activated β-catenin polypeptide.

18-19. (canceled)

20. A set of isogenic cell lines, comprising:
a first population of cells that are null for β-catenin expression, and
at least a second population of cells that express a wild type β-catenin polypeptide, wherein the wild type β-catenin polypeptide functions as an activated β-catenin polypeptide,
wherein at least one of the populations of cells contains a disrupted β-catenin gene, and
wherein, except for a nucleotide sequence of the β-catenin gene, the first population of cells and the at least second population of cells are substantially genetically identical.

21. The set of isogenic cell lines of claim 20, wherein the cells of the second population of cells are homozygous for a wild type β-catenin gene, or wherein the cells of the second population of cells are hemizygous for a wild type β-catenin gene.

22-27. (canceled)

28. A recombinant nucleic acid molecule, comprising at least a first polynucleotide having a first end and a second end, wherein the polynucleotide is flanked at the first end by a first nucleotide sequence of a β-catenin gene and is flanked at the second end by a second nucleotide sequence of a β-catenin gene,
wherein the polynucleotide is heterologous with respect to the β-catenin gene,
wherein the first and second nucleotide sequences of the β-catenin gene are different from each other, and
wherein each of the first and second nucleotide sequences of the β-catenin gene can specifically hybridize to a β-catenin gene under physiological conditions.

29. The recombinant nucleic acid molecule of claim 28, wherein the first and second nucleotide sequences of the β-catenin gene specifically hybridize to a wild type β-catenin gene or to a mutant β-catenin gene, which encodes an activated β-catenin polypeptide, or
wherein the first and second nucleotide sequences of the β-catenin gene specifically hybridize to both a wild type β-catenin gene and a mutant β-catenin gene, which encodes an activated β-catenin polypeptide.

30-40. (canceled)

41. A vector, comprising the recombinant nucleic acid molecule of claim 28.

42. (canceled)

43. A method of producing a set of isogenic cell lines, which comprises a first population of cells that express a wild type β-catenin polypeptide and at least a second population of cells that express an activated β-catenin polypeptide, the method comprising:
a) introducing a recombinant nucleic molecule of claim 28 into cells that are heterozygous for a mutant β-catenin gene, which encodes an activated β-catenin polypeptide, and a wild type β-catenin gene, which encodes a wild type β-catenin polypeptide; and
b) selecting
a first population of cells derived from a cell containing the recombinant nucleic acid molecule integrated into only the mutant β-catenin gene, wherein said cells express the wild type β-catenin polypeptide, and
at least a second population of cells derived from a cell containing the recombinant nucleic acid molecule integrated into only the wild type β-catenin gene, wherein said cells express the activated β-catenin polypeptide,
thereby producing a set of isogenic cell lines, which comprises at least a first population of cells that express a wild type β-catenin polypeptide and at least a second population of cells that express an activated β-catenin polypeptide.

44. (canceled)

45. The method of claim 43, wherein the polynucleotide in the recombinant nucleic acid molecule encodes a polypeptide, and wherein, optionally, the polypeptide is neomycin acetyltransferase.

46-53. (canceled)

54. A set of isogenic cell lines produced by the method of claim 43, said set of isogenic cell lines comprising a first population of cells that express a wild type β-catenin polypeptide and at least a second population of cells that express an activated β-catenin polypeptide.

55-56. (canceled)

57. A method of identifying a therapeutic agent that allows selective killing of cells expressing an activated β-catenin polypeptide, the method comprising:
a) contacting the isogenic set of cells of claim 1 with at least a test agent to be examined for therapeutic activity; and
b) detecting selective killing of the cells expressing the activated β-catenin polypeptide as compared to the cells expressing the wild type β-catenin polypeptide, thereby identifying a therapeutic agent that allows selective killing of cells expressing an activated β-catenin polypeptide.

58. The method of claim 57, wherein the therapeutic agent selectively kills the cells expressing the activated β-catenin polypeptide.

59. The method of claim 57, further comprising contacting the set of isogenic cell lines with a toxic agent, and identifying a therapeutic agent that protects the cells expressing the wild type β-catenin polypeptide from the toxic effect of the toxic agent, thereby allowing selective killing of cells expressing the activated β-catenin polypeptide.

60-61. (canceled)

62. The method of claim 57, wherein the test agent comprises one of a plurality of test agents.

63-65. (canceled)

66. The method of claim 57, which is performed in a high throughput format.

67-70. (canceled)


SEQUENCE LISTING (1 sequence)

SEQ ID NO: 1 - DNA, 8850 bp, Artificial sequence
  Recombinant nucleic acid molecule containing a polynucleotide sequence encoding a neomycin resistance gene flanked by nucleotide sequences of the human beta-catenin gene
aagcttcactgtatgtatgatcttgttgactctcttactcattgttattgtaataccagtaacaaagggtgtttaaaatttgttcagtgggtgaatatatgttccatttaatggataaattattttttattcagtctcctgttgatggacatttgaataatttccatctttttctctatgaatgcctcacttggcatgcttctgacagtattgccacagaatacatttctgttataaaaattgaatttttaagtcaaagggtagttacactttaatggatagtggcagcttactatcaaaagtttctgctagtttcaccatatccttattagcagtagatattatcaatcttttcaatctttgccaatctgataagcaaaaagtaaatgggtttaaacatcctttgtatatattcattgctcactttatgtttttcctttgaaatgttatttcttgttctttccctgcagtatgattctttctttttttgacttgttcccagttttttgtgtactatggatattagcctttaattatgttacggatgttctagtatgttattttttgaattacttcaaatgtgatttgttgctcagattttaaaaactacatacacaaattatctcatgtttccctttttggtttcaatttcgactcatgcttaatcagttcatcgattgggcatggttttattcttaatatatacccgtattttatctcattttatttttttacgtgtaaatatttggtgaatataggtttaattttaatgtaaaataaggatgaaaaatgatagttggaattacaagcccatttctcctaatacttttaatcaagtaatccactaattgaaatattaccttcttcatttatgaaattgccacattatatctgggtgtttttctgcctactacagtctcttacccatttctttcctaataatacaatacttgaattgctgtggttgttgatttataatgttatcttaatgataacattataaatgtgatggaactggttcctccttatagttcttcttaaatcaagaacaagacatatcttcccatttactctcgtatgtatctcattttactgttatgaatgaaatctgtcctatttgtgtataggaaaatagtttttgtatgtaattgtgatatggccagttttattaaaaatttggttaaactaagagttgttttctgttcagccttatcatactataaaatccacataaaatgggtataaaagtgtcgcaggacactgggctcagatgattctcccacctcagcttcccaagtagctgggactacagcggcatatgccaccacacccagccaatttttaaataagttttaaaaatagtatttttagtagagacagggtttcaccatgttgcccaggctggtcttgaactcctggactcagacaatccacctgccttggcttcccaaagtgttgggattacaggtgtgagccaccacaccttgccgaattgcagccatatttaatacttttttccatcctattccctttgctgcccccaggcctcctgtattgatagcccgctattaagaagctagtgtatattctttgcatacttttacttcataaactatatgaagcattgttctgttttttaacttaattggtataaaattatattttggaaattcagtatattctgtgaaaattatttagaaaatgtgcctctgagataaagcctattcaggatgtatcttaaaggagatagctgtgctttaacattatcagtctttttggctgcttatgttaatataagttggagaaaaacagtctgctttttgtgataatatgttcttggagatggagtgaaagattgtttaaaaacattgtcttttttttcccctgaagtaccagtatttattttaggattatgttactgatcaaagatgctgtgtggagttactcattggtgagactaacaataaatcacacatgcaaaggatgttaccataatctaattattttaaacagtaaaattatattctaagacatccagttggcctatatgtgctatatcaatgactatcaaggggctttttatgtatactgtatacatgtacttcacaaaaatataaaaggatgacatcaaaaatctggcaagccaaaagcctacattacatgtagcaaataaataagcatatgaacttattggaatttaaaaccctgtaggatgggcgggtgatggtatgtatgttagatgtgtggacatatctattaaaagttgtgtcagataacagctggtgctgacaagcccttggtaagatggcagcatgttcaatatgttctgtgaaaattatctcagtttatgatctgtcagtattgtggagctatgcatgaaaggacttaaaattcttacccttaaactcagtaacagtgtttctagaacttctggtgatatgggaaattaagagaattatttatatgcaaaggtgtttattgcagcattgttggaataatagacaaaatggggaagaacaagctcagaatggaggaggtagcttatagtatagacatacgatacaatccagatgataatattttataatagtcttcacaaggaattttatatttttatttttaaaaatacatagcaagtgagtttaatataccaaacataccaaaatgtcatcatttactgtgtggtggactcatatgatggagatgataaataaaaatattaatttatttgaggcatatatttatggctgaggaaggaagacagttatgaagaacagctcattctggaaacatactaatttttcccagccataaagagatttcctatttcttttttttttccatttaccttctgtttcctacctgagaagatttcatacttctaataaccatttgtgtacctatttaaagacagtaccaaaggcatacattttagtgtttggaggaccaagggtcatttgatgtttgatgcttattgactattcgaggatgacaagacaccttgagaacacacacacccacacccacacccacaccctcacccacccaccccacccccctccccgaagaaagctgtgaaggaagaaagcagaaaagaacctggagtgagttgtaacttaaagtgttagtgttgcatgaagtgtgttaaaacaggaagatttgaggaaattgcatacattttctagatggcaaagtattactggtgacagttaatgaaaatgcatatgcatgtgtttttagatttacaaattttactaagaactttttaaaaatccctgaaggtgtatcaaaagtttatcatgcttatgaaatagagtagcactttctaactttaaaacggggaataattctttggatcttgattattggaaaagtgaattatgaattgctagtataaaactgtggttttaaaatatgtctgctttatatttttatgtagcagatttactcctagttaataatactcaaacttactgaaaactaaggtaattaagataattctgtcctgatgggaagaggaaaaataacttcagtgtgaaatctattatatattagttgtggcaagatttctcccattgactttgactggagacatttatagggttaaaatcggaaatagcacggtgaattttgaagtatccttgtagttggaaagagtattatgttcatattgccaaaaaaaagatgcatggatgcattagactggatggaaaatacatgagaagttggctagccccctctttgtcaaaacatcacttggtggtgataaagctgttggaaaacacagcattctaatgtagtctgtagtttaatgataatctgtgtcttgaaacatttagcgtagtacttatacaaacctagatggcatagtgtactgcatgcctagcctatatagtatagcctgttgcttctagggtgtaaagctgtatagcgtgttactataggcagttgaaacagtggtatttatgtatccttttttttttttttaaattcttttaagagacagggtcttgctctgttgcccaggctggatgcattggtgtgatcatagctcactataaccttgaactcctaagtgatcctctttgcctcagcctccccagtggctaggactacaggcacatactaccacacctggctaatttttaacatttttttgtagagatggaatttcgctgtgttgtccaggctggtcttggaactcttgtgctgcagcaatccacccgcctcccaaagtgttagaattacaagccacttcgcctggcttgtttacctaaacatagaaaagatccagtaaaaatacagaattaaaatcttgtggggccactgtagcatatgtagtccatcttgactgaaatgtccttatgcagtgcatgattgtacttcataatttttaagcactcctccctcttgattggtacttagtggattttatcatttttgtttcttcataattctttctgaaatgtctactggttggacctttgatctcctgaattgatcgtgatttcttctgttgtattttttgtctttgtcatttttttgtactctaggcagttttctcaattttagtttctattcaactttttgtttttatttattctctccagtatttatggagatactaaattgaagtgttctgtttctctctccaccctatccctagtttcaagttttatctcagtttctatggagtcagttttttcgttgctttaaaaaaaaattttcctgaagtgattggtaagttttggctaattgggagcactagaattgggcccttaatggttggcagggtgtggtggaggagagacagcccttagtccaaaggctcaggccagaaaaagaaagaggaaggctttccttttcctttccggagcagggttctgccctaggtcttgcttggcagtctatttgatttctttagcagttaatgctcagttttttggcatatgtggatctgcctccagagcaggtacaaggtgagtgagtctatgctgttacctaattagatccccatttctaccctttgtttttacttctctatctactgataggtttttaccctccttcacctcatagggttgcagtgaagagcaagatgaatttttatttatgttgcataaattttaaaagctaaaaaatatatatgtaatgttgggaagtcccagtgtacaaatggctattgtaaatttggaacatgaacttgcttttttccattgtaaaaatgaaatcattataaattgcgggtcaagttactaggtcagcccacacagagtttacccagtaatatgcgtaaatgttttgcctttgcatcaacaacaaggaaaaacagtactataaaaaaatgttcctggaagccggatgtatcaaagcacttctgaaatagctatatagcctatagacatgaccagttggtttctgagtctgttgacattggccaaaggagaagctcagtgtagaacatgtttggagtctccttttgcagaaatacattggaggctggagtggggaaccaatttttcagaaaggtggtgaagtagttacatagccactcttttaaagacagtcaaaagatagaaactaaggccaggtgttggctcacatctgagataggaaaatcacttgaacctgggaggcggaggttgcagtgagcccagtatgcacctctgcactccagcctggtttggcaagagaccaaaactctgtctcaaaaaaaaacaaaacatagttcacacttaaatattttattccatatctttacatacccaatatgttaatttatagttcaagatgaacttgtttgggacagattttgtaataaaggaaatcgtgttattagaaatatctagaggccatgagcccttaaactgttctaatttgcaagtagttccctgtgtgatgcagtttttttcaatattgcacaataaaggcaaaatacggacaaattagatgataagatttatataaatttttaaaatattgatcaaaatatgtatccatattggtaatatttgtatttataataaatcattgctgtaaatttgaacttagaaaaattttactaataaaggtgcttttgtgttgcaaactttcatttgaaaagtaatttttctttgtaccaaaaaatctaaaattcgctattctagtcaccaaaatttgctttatgaaaaataatttttgatggcactatatcagaaaacaacttgttaaagaaaatgtggagtttttaaaatcccactgtacctctgttatccaaaggggatctgtgaatttttctgtgaaaggttaaaaaaggagagacctttaggaattcagagagcagctgatttttgaatagtgttttcccctccctggcttttattattacaactctgtgctttttcatcaccatcctgaatatctataattaatatttatactattaataaaaagacatttttggtaaggaggagttttcactgaagttcagcagtgatggagctgtggttgaggtgtctggaggagaccatgaggtctgcgtttcactaacctggtaaaagaggatatgggttttttttgtgggtgtaatagtgacatttaacaggtatcccagtgacttaggagtattaatcaagctaaatttaaatcctaatgacttttgattaactttttttagggtatttgaagtataccatacaactgttttgaaaatccagcgtggacaatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatccgtcgactttcatcactgagctaacccagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtaagcaatgacatagctagctttttagtctgctttgaagtaaatgctcaaggggagtagtttcagaatgtctacccaataccagtacttgaaaactaacgatgtttctgaattcctgtattacaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttgggtaagaaaacatgtcagaatgcttgaagctaaaaagtagaagagtatactcacaatatttctgatgaggcttttttcttcttcccagttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaaggtaagagaattattctttatgtggttttcatggagcattggacacctccagtgtcatgtcattccatgcagtgttcctaacctttttggcaccagggaccagtttcgtggaaaacagtttttccatgaatgggttgtgggaatggtttctggatgacaccattccacctcagataatcaggcattagattctcatagggagcgtgcagcctagatccctcgcatgtgcagtccacactagggtttctactcctatgagactctcatggtgcagttgatctgacaggaggtagagctcaagccaggtaatgctcgctcacctgccacttacctcctgctgtgcagcccagttcatttctgttcttttaaatttttgagtttccatatgtaaagcactatgcgaagtagtagggatatggtaggcgtcgac